(19) United States
(12) Patent Application Publication
(10) Pub. No.: US 2025/0108230 A1
(43) Pub. Date: Apr. 3, 2025

(54) HAND-HELD LIGHT-GUIDE-COMPRISING APPARATUS FOR EMITTING INTENSE PULSED LIGHT

(71) Applicant: QUANTEL MEDICAL, COURNON-D'AUVERGNE (FR)

(72) Inventors: Benjamin WASSMER, CLERMONT FERRAND (FR); Thomas SECOND, SAINT-FARGEAU (FR); David PUREUR, CLERMONT FERRAND (FR)

(73) Assignee: QUANTEL MEDICAL, COURNON-D'AUVERGNE (FR)

(21) Appl. No.: 18/730,222

(22) PCT Filed: Jan. 19, 2023
(86) PCT No.: PCT/FR2023/050074

§ 371 Date: Jul. 18, 2024

(30) Foreign Application Priority Data
Jan. 19, 2022 (FR) FR2200450

(51) Int. Cl. A61N 5/06

(52) U.S. Cl. CPC A61N 5/0616; A61N 2005/063; A61N 2005/0663; A61N 2005/0667

(57) ABSTRACT

The invention relates to a hand-held apparatus for emitting intense pulsed light comprising a body configured to be hand held, said body having an aperture accommodating a treatment interface (8) configured to be placed against a skin, the body housing a lamp (14) suitable for emitting a light pulse through the treatment interface in an emission direction, the treatment interface comprising a light guide (10) that protrudes from the body of the hand-held apparatus, the light guide (10) forming a polyhedron having end faces (10a) that are opposite in the emission direction and side faces (10b) joining said end faces, the light guide (10) having a thickness, between the end faces (10a) in the emission direction, larger than 6 mm, and the side faces (10b) having an arithmetic mean roughness Ra lower than or equal to 3.2.

### TECHNICAL FIELD

The present invention belongs to the field of photo-stimulation systems of the human body, and more precisely relates to a hand-held light-guide-comprising apparatus for emitting intense pulsed light, in particular for ophthalmic applications.

### STATE OF THE ART

The principle of intense pulsed light (or “intense Light Pulse”, IPL) is based on the emission of light towards the skin. An intense pulsed light system comprises a console and a hand-held apparatus. The operator adjusts the parameters of the light pulse (duration, intensity, etc.) on the console then places the hand-held apparatus in contact with the area of the patient to be treated, and finally triggers the light shot. Light interacts with components of the skin or subcutaneous components, depending on the wavelengths. The impact time of the light pulse can vary from 1 ms to 100 ms.

The hand-held apparatus for emitting intense pulsed light thus comprises a body configured to be hand held, said body having an aperture accommodating a treatment interface configured to be placed against a skin. It is possible to apply a gel to ensure good contact and light transmission.

However, current hand-held apparatuses for emitting intense pulsed light are not entirely satisfactory. Indeed, they suffer from a lack of precision, the current configuration not allowing the operator to correctly visualize the treated area. In addition, the uniformity of light emission over the treatment area is generally low.

Such limits are not problematic when the hand-held apparatus for emitting intense pulsed light is used for applications not requiring great precision, such as for example skin aesthetics, mainly permanent or semi-permanent hair removal (photo-hair removal), semi-permanent hair removal, or treatment of signs of aging such as pigment spots (photo-depigmentation). On the other hand, applications such as healthcare may require more precision in the treatment area, and current hand-held apparatuses are not suitable for this. This is for example the case for ophthalmic applications such as light stimulation of the tear ducts, where the treated area must be precisely located under the eye. If it is too far from the eye, the stimulation will not cause the expected effect, whereas, conversely, being too close to the eye can be dangerous. It then becomes even more important that the entire treated area receives an adequate amount of light, since there can be no question of moving the hand-held apparatus to smooth out inhomogeneities.

### PRESENTATION OF THE INVENTION

The purpose of the invention is to provide a hand-held apparatus allowing increased spatial precision to homogeneously illuminate an area of the skin to be treated.

For this purpose, a hand-held apparatus for emitting intense pulsed light is proposed comprising a body configured to be hand held, said body having an aperture accommodating a treatment interface configured to be placed against a skin, the body housing a lamp suitable for emitting a light pulse through the treatment interface in an emission direction, wherein the treatment interface comprises a light guide that protrudes from the body of the hand-held apparatus, the light guide forming a polyhedron having end faces that are opposite in the emission direction and side faces joining said end faces, the light guide having a thickness, between the end faces in the emission direction, larger than 6 mm, and the side faces having an arithmetic mean roughness Ra lower than or equal to 3.2.

The apparatus is advantageously completed by the following characteristics, taken alone or in any of their technically possible combinations:
the light guide has an end face having a surface extending along a short axis comprised between 15 mm and 30 mm, and a long axis comprised between 30 mm and 60 mm;
the light guide protrudes at least 5 mm relative to the body;
the side faces have an arithmetic mean roughness Ra greater than 0.2.
a surface condition of the side faces results from cutting the light guide from glass;
the hand-held apparatus further comprises an opaque cover surrounding the side faces;
the light guide is formed of a material having a refractive index greater than 1.45;
the hand-held apparatus comprises a filter disposed between the lamp and the light guide, configured to filter at least wavelengths less than 580 nm, the filter and the light guide being formed of different materials;
the light guide is formed of a glass block.

The invention also relates to an intense pulsed light system comprising a hand-held apparatus according to the invention, and a console to which said hand-held apparatus is connected.

### PRESENTATION OF THE FIGURES

The invention will be better understood, thanks to the description below, which relates to embodiments and variants according to the present invention, given by way of non-limiting examples and explained with reference to the appended schematic drawings, in which:

FIG. 1 shows in a simplified manner a hand-held instrument according to a possible embodiment of the invention:

FIG. 2 schematically shows an illumination assembly comprising a lamp and a light guide according to a possible embodiment of the invention;

FIG. 3 shows the whole of FIG. 2 from a different angle;

FIG. 4 schematically shows an illumination assembly comprising a lamp, a light guide and a cover according to a possible embodiment of the invention

FIG. 5 shows the whole of FIG. 4 from a different angle;

FIG. 6 is a graph illustrating the distribution of the luminous flux along a long axis at the outlet of the light guide, according to several configurations of the hand-held apparatus;

FIG. 7 is a graph illustrating the distribution of the luminous flux along a short axis at the outlet of the light guide, according to several configurations of the hand-held apparatus.

### DETAILED DESCRIPTION

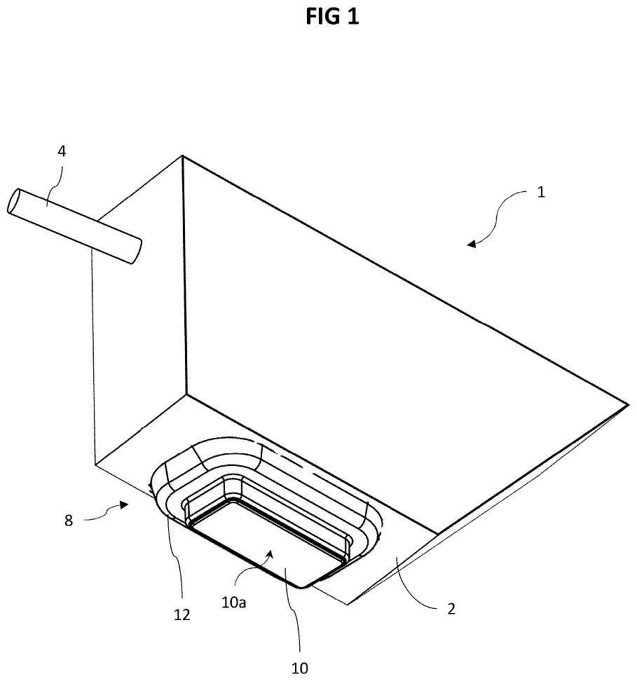

With reference to FIG. 1, the hand-held intense pulsed light apparatus 1 comprises a body 2 configured to be hand held. The hand-held apparatus 1 is typically connected to a console, and therefore comprises a wired connector 4 allowing the supply of electricity to the hand-held apparatus 1. The console integrates all the electronics necessary to charge and discharge high voltage capacities (300 to 1000 V typically). The wired connector 4 can also integrate water circulation between the console and the hand-held apparatus 1 for cooling purposes. The console also integrates adjustment means allowing to adjust the parameters of the light pulse (duration, intensity, etc.).

The body 2 has an aperture accommodating a treatment interface 8 configured to be placed against a skin, that is to say in contact with this skin or at least very close to it (less than 1 cm). The treatment interface 8 comprises a light guide 10 that protrudes from the body 2 of the hand-held apparatus 1, in the emission direction. Preferably, the light guide 10 protrudes at least 5 mm relative to the body 2, and preferably at least 7 mm. The projection of the light guide 10 means the height to which the light guide 10 rises relative to the wall of the body 2 which surrounds the light guide 10 in the emission direction.

In this example, the treatment interface 8 comprises a cover 12 surrounding the light guide 10, in particular at the periphery of the aperture of the body 2. The cover 12 leaves a face 10a of the light guide 10 free in a direction of emission of light, which is the face 10a intended to be placed against the skin.

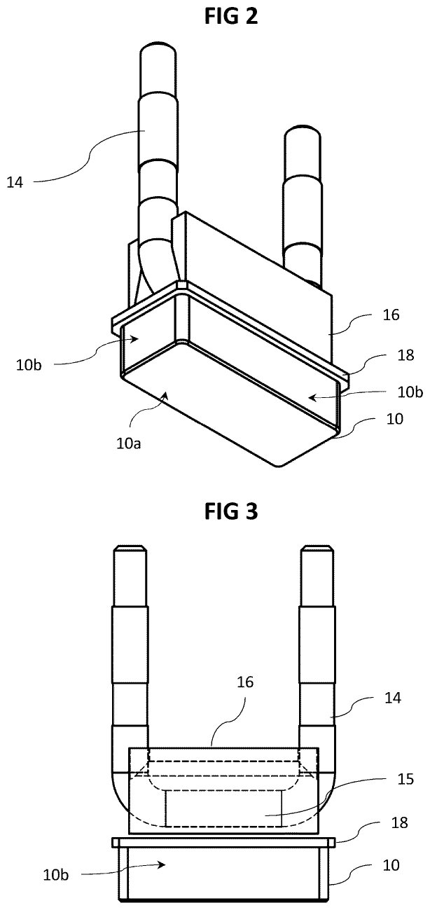

With reference to FIG. 2 and FIG. 3 illustrating an illumination assembly, the body 2 of the hand-held apparatus 1 houses a lamp 14 configured to emit a light pulse through the treatment interface 8 in an emission direction. The lamp 14 is preferably a flash lamp. When emitting a light pulse, a high voltage is discharged through the lamp 14, emitting high energy light (10 to 200 Joules) for a very short time (1 to 10 ms) and over a wide wavelength spectrum (400 to 1200 nm). In the example illustrated, the lamp 14 takes a U shape, the part which emits the useful light being located in the junction 15 of the branches of this U, at which a reflector 16 surrounds the lamp 14 except in the emission direction. The reflector 16 allows to return rays which would otherwise heat the body 2 of the hand-held apparatus 1 to the output of the treatment interface 8. Typically the reflector 16 is an opaque, reflective or at least white part in order to reflect light, and preferably very diffusing in order to homogenize the returned light. Preferably, the hand-held apparatus 1 is configured to accommodate a circulation of water in contact with the lamp 14, and preferably between the reflector 16 and the lamp 14.

The illumination assembly typically comprises a filter 18 disposed at the outlet of the lamp 14 in the direction of illumination, to reduce the spectrum of the light emitted depending on the applications. Preferably, such a filter 18 is configured to filter the wavelengths of the light pulse less than 580 nm (cut-off frequency or transmittance less than 0.1), and preferably less than 600 nm. For example, the filter 18 is a SCHOTT RG-610 high-pass filter with a cutoff frequency of 610 nm. Filtering low frequencies allows to reduce the sensitivity of light stimulation to skin tone. Typically, the filter 18 is planar and has a thickness of 1 to 4 mm, preferably less than 3 mm.

A light guide 10 is disposed after the filter 18 in the emission direction. The light guide 10 forms a polyhedron having end faces 10a that are opposite in the emission direction and side faces 10b joining said end faces 10a. Typically, the light guide 10 is a hexahedral prismatoid with quadrilateral faces, and more precisely a cuboid such as the right block illustrated. The light guide has a thickness, in the emission direction, larger than 6 mm, and preferably larger than 8 mm, and even more preferably larger than 11 mm. The thickness must be sufficient to allow uniform distribution of light over the area to be treated. However, it should not be too long so as not to reduce the transmitted energy too much. Preferably, the thickness is less than 30 mm.

For example, the end face 10a may have a surface extending along a short axis comprised between 15 mm and 30 mm, and a long axis comprised between 30 mm and 60 mm.

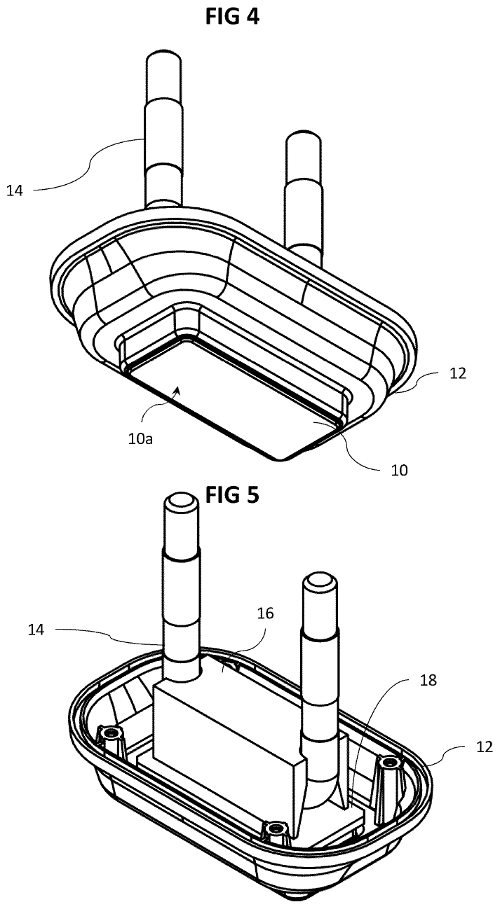

As illustrated in FIG. 4 and FIG. 5, the cover 12 can cover the side faces 10b of the light guide 10. The cover 12 allows protection against a danger on the light diffused by the side faces 10b of the light guide 10 without modifying either the final transmitted energy or the transverse profile of the transmitted light. In fact, the cover 12, or at least part of it, protrudes relative to the body 2 of the hand-held apparatus 1 in the emission direction, preferably on the same height as the light guide 10. Part of the cover 12 is housed in the body 2.

The light guide 10 is made of a transparent material, capable of allowing illumination energy to pass through. Typically, the light guide 10 is formed in one piece. Preferably, the light guide 10 is made of glass, and for example of borosilicate glass such as N-BK7 or a glass called dense flint glass such as SF11 from Schott. Preferably, the light guide has a high optical index, that is to say a refractive index greater than 1.45, preferably greater than 1.51, and preferably greater than 1.60, and preferably still greater than 1.70. Having a glass with a high optical index allows for greater total internal reflection, and therefore allows for better guiding of the light in the emission direction to reduce energy losses and avoid having an emission light through the side faces 10b, which could be dangerous. Preferably, the filter 18 and the light guide 10 are made of different materials.

In order to ensure not only uniform treatment of the area to be treated, but also so that it can be ensured that the entire area receiving the light receives enough light to guarantee precision in the location of the treatment, it is important to ensure that the transverse emission profile at the output is as homogeneous as possible. In this regard, the surface condition of the side faces 10b of the light guide can be used to improve the constancy of the transverse emission profile.

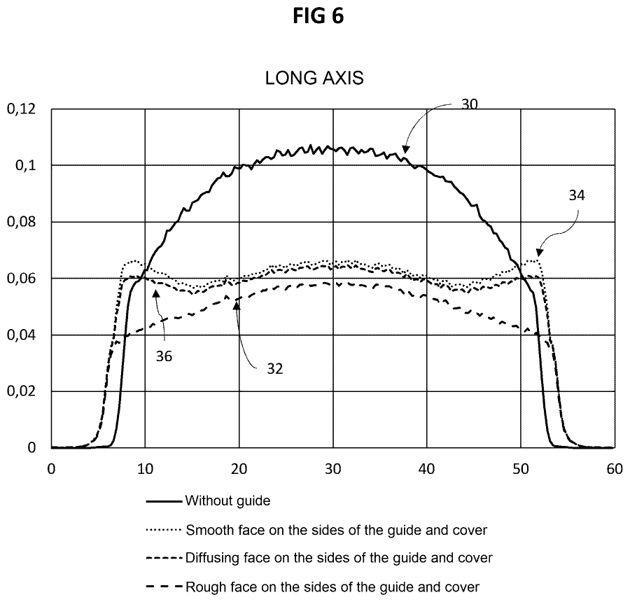

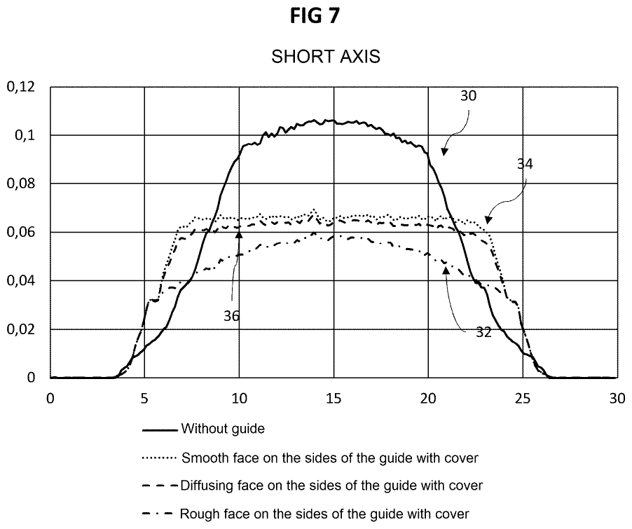

FIG. 6 shows the distribution of the luminous flux along a long axis at the outlet of the light guide, according to several configurations of the hand-held apparatus, while FIG. 7 shows it along a short axis. The abscissa axes are in millimeters, while the ordinate axis is an arbitrary measurement representative of the average intensity, on an axis perpendicular to the abscissa axis, of the energy received in a reception zone.

The first curve 30 in solid lines corresponds to the transverse emission profile of a hand-held apparatus 1 without light guide 10 or cover 12. The second curve 32 in dashes and dots corresponds to the transverse emission profile of a hand-held apparatus 1 with a light guide 10 and cover 12, the light guide 10 having rough side faces 10b. The third dotted curve 34 corresponds to the transverse emission profile of a hand-held apparatus 1 with a light guide 10 and cover 12, the light guide 10 having smooth side faces 10b. The fourth dashed curve 36 corresponds to the transverse emission profile of a hand-held apparatus 1 with a light guide 10 and cover 12, the light guide 10 having diffusing side faces 10b. It should be noted that the presence or absence of a cover 12 does not change the results, a configuration with light guide 20 but without cover 12 has not been shown. In all configurations, the shapes and materials of the light guide 10 are the same (block of 47 mm by 19 mm in BK7).

It is first noted that a hand-held apparatus 1 without a light guide 10 has a transverse emission profile (first curve 30) varying enormously with the abscissa, both on the long axis and on the short axis, with a bell shape, which is not desirable. A hand-held light-guide 10 comprising apparatus 1 with rough side faces has a transverse emission profile (second curve 32) which varies less, but which takes significantly lower values, indicating a significant loss of energy. A hand-held light-guide 10 comprising apparatus 1 with smooth side faces has a transverse emission profile (third curve 34) which is practically flat and with higher values than those obtained with rough faces. A low roughness of the side faces therefore allows to increase both the constancy of the transverse emission profile and the amount of energy transmitted, thus limiting losses.

It appears, however, that the side faces do not need to be necessarily smooth for the light guide 10 to have satisfactory characteristics. A hand-held light-guide 10 comprising apparatus 1 with diffusing side faces has a transverse emission profile (fourth curve 36) with qualities similar to those obtained with smooth faces, both in flatness of the transverse emission profile and in amount of energy transmitted.

A smooth face is obtained by polishing this face after cutting the material of the light guide 10, and therefore has a roughness with an arithmetic average deviation Ra lower than or equal to 0.2. A diffusing side face is obtained by retaining the surface condition after cutting the material of the light guide 10, or with minimal polishing, and has an arithmetic mean roughness Ra lower than or equal to 3.2. A rough face designates a face with an arithmetic mean roughness Ra strictly greater than 3.2. Such roughness is for example obtained by means of a surface treatment of the side faces 10b such as roughening or sandblasting.

Thus, the use of a light guide 10 with side faces having an arithmetic mean roughness Ra lower than or equal to 3.2 allows to obtain the desired qualities of precision. To the extent that each intervention involves costs, it is preferable to use a light guide 10 made of cut and raw glass without polishing or treatment since polishing does not allow to obtain gains justifying the additional costs. Consequently, the side faces 10b preferably have an arithmetic mean roughness Ra greater than 0.2, and preferably greater than 0.5. Preferably, the surface condition of the side faces results from cutting the light guide 10 from glass.

The invention is not limited to the embodiment described and represented in the appended figures. Modifications remain possible, particularly from the point of view of the constitution of the various elements or by substitution of technical equivalents, without departing from the scope of protection of the invention.

1. An apparatus, the apparatus being a hand-held apparatus for emitting intense pulsed light, the apparatus comprising a body configured to be hand held, the body having an aperture accommodating a treatment interface, the treatment interface being configured to be placed against a skin, the body housing a lamp suitable for emitting a light pulse through the treatment interface in an emission direction, wherein the treatment interface comprises a light guide that protrudes from the body of the hand-held apparatus, the light guide forming a polyhedron having end faces, the end faces being opposite to each other and orthogonal to the emission direction, the polyhedron having side faces, the side faces joining the end faces, the light guide having a thickness larger than 6 mm, the thickness being defined between the end faces in the emission direction, and the side faces having an arithmetic mean roughness Ra lower than or equal to 3.2.

2. The hand-held apparatus according to claim 1, wherein one of the end faces presents a first length along a short axis and a second length along a long axis, the first length being comprised between 15 mm and 30 mm, and the second length being comprised between 30 mm and 60 mm.

3. The hand-held apparatus according to claim 1, wherein the light guide protrudes at least 5 mm relative to the body.

4. The hand-held apparatus according to claim 1, wherein the side faces have an arithmetic mean roughness Ra greater than 0.2.

5. The hand-held apparatus according to claim 1, wherein the light guide is formed from cut and raw glass without polishing or treatment.

6. The hand-held apparatus according to claim 1, further comprising an opaque cover surrounding the side faces.

7. The hand-held apparatus according to claim 1, wherein the light guide is formed of a material having a refractive index greater than 1.45.

8. The hand-held apparatus according to claim 1, comprising a filter disposed between the lamp and the light guide, the filter being configured to filter at least wavelengths less than 580 nm, the filter and the light guide being formed of different materials.

9. The hand-held apparatus according to claim 1, wherein the light guide is formed of a glass block.

10. An intense pulsed light system comprises a hand-held apparatus according to claim 1, and a console to which said hand-held apparatus is connected.
